(19) United States
(12) Patent Application Publication
(10) Pub. No.: US 2023/0354785 A1
(43) Pub. Date: Nov. 9, 2023

(54) Autonomous Biomonitoring System in Lotic Ecosystems

(71) Applicant: Baylor University, WACO, TX (US)

(72) Inventor: Ryan A. MCMANAMAY, China Spring, TX (US)

(73) Assignee: Baylor University, Waco, TX (US)

(21) Appl. No.: 18/312,182

(22) Filed: May 4, 2023

Related U.S. Application Data
(60) Provisional application No. 63/339,251, filed on May 6, 2022.

(51) Int. Cl. A01K 61/95; G01N 1/20; G01N 35/00; G01N 33/18

(52) U.S. Cl. CPC A01K 61/95; G01N 1/2035; G01N 35/0099; G01N 33/1886

(57) ABSTRACT

The present disclosure provides an autonomous biomonitoring system in lotic ecosystems, such as streams. A central design concept of autonomy is capturing aquatic organisms from their natural habitat and entraining them through an analyzer with an imaging unit. The system provides a collector for a sampling aqueous mixture that can flow through a screen to a sediment trap that is coupled to a pump that discharges the aqueous mixture into a staging tank. The staging tank is coupled to a flow-through analyzer and can continuously buffer and control the volume of aqueous mixture flow through the analyzer, and then the aqueous mixture is discharged. In at least one embodiment, the system allows continuous monitoring over a period of time. The system can transmit real time data to a remote location for data accumulation. The system can further provide remote movement of the collector for sampling at multiple remote locations.

### CROSS REFERENCE TO RELATED APPLICATIONS

This application claims the benefit of US. Provisional Application Ser. No. 63/339,251, entitled “Autonomous Biomonitoring System in Aquatic Environments”, filed May 6, 2022, which is incorporated herein by reference.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

### Field of the Invention

The disclosure generally relates to biological sampling and monitoring systems in lotic ecosystems, that is, ecosystems have moving water, such as a run, creek, brook, river, spring, channel or stream. More specifically, the disclosure relates to portable, user-friendly, autonomous biological sampling and monitoring of organisms, including macroinvertebrates, in lotic ecosystems and associated methods of use.

### Description of the Related Art

Sample collection for small aquatic organisms (1 mm-50 mm), including benthic macroinvertebrate and larval fish, and subsequent sample processing, is a notoriously labor-intensive and resource intensive enterprise that requires taxonomic expertise. Additionally, accurate taxonomic identification requires preserving samples, leading to mortality of such organisms. As such, methodological paradigms for such collection and subsequent laboratory sample processing have largely remained stagnant for the past several decades, which, ultimately, has resulted in limited spatiotemporal richness of biomonitoring information relative to other recent technologies that provide higher volumes of data (e.g., remote sensing, water quality).

Typical stream biological sampling, including sampling of such organisms, is performed in individual samples using a Hess sampler. The Hess sampler has been used for several decades as the standard for streams sampling. The Hess sampler is a metal cylinder, analogous to a stove pipe with openings on upper and lower ends and at least one upstream window and at least one downstream screened window in the sides of the sampler. The downstream window is coupled to a downstream mesh tapering to a collection container. The lower open end of the Hess sampler is typically pushed into the stream substrate, such as mud or gravel, typically 3-6 inches to disturb the substrate and to prevent escape of organisms outside the Hess perimeter. The flow of current carries target organisms as well as sediment and debris entrained in the aqueous mixture and the disturbed substrate into the Hess sampler, then through the downstream net and into a collection bucket. The net is constructed of mesh in a choice of sizes. The Hess sampler yields a single collection of a sample to be returned to a laboratory for examination. The Hess sampler is the standard accepted device used by research institutions, government agencies, and the private sector. The advantages of the Hess sample are light weight, portable, allows access to remote streams. The disadvantages are single sample per unit, single point of sampling, and single time of sampling. The Hess sampler has no real time data and therefore no real time monitoring.

Therefore, there is a need for an improved biological monitoring in lotic ecosystems that can provide at least multiple samples and preferably continuous sampling.

### BRIEF SUMMARY OF THE INVENTION

The present disclosure provides an autonomous biomonitoring system in lotic ecosystems, such as streams, for small aquatic organisms (1 mm-50 mm), including benthic macroinvertebrates. A central design concept of autonomy is capturing organisms from their natural habitat and entraining them through an analyzer with an imaging unit. The system provides a collector for an aqueous mixture that includes water and potential aquatic organisms that can flow through a filter to a separator that is coupled to a pump that discharges aqueous mixture into a staging unit with a staging tank. The staging tank is coupled to a flow-through analyzer that includes a scanner, electronic processor, and a database of characteristics of target aquatic organisms to analyze the aqueous mixture as it flows through the analyzer to determine types and quantities of any present aquatic organisms. The mixture can be discharged into the lotic ecosystem. In at least one embodiment, the system allows continuous monitoring over a period of time. The system can transmit real time data to a remote location for data accumulation. The system can further provide remote movement of the collector for sampling at multiple remote locations. The autonomous biomonitoring system provides data collection that heretofore has been unrealized as a significant shift in paradigm for sampling of streams. The invention can provide sampling in the field of aquatic organisms, particularly benthic macroinvertebrate and larval fish sampling, sample processing, and sample identification towards autonomy to provide higher volumes of information that is more representative of the actual conditions in the lotic ecosystem.

The disclosure an autonomous biomonitoring system for organisms in a lotic ecosystem, comprising: a collector configured to collect an aqueous mixture having aquatic organisms; a pump coupled to the collector and configured to flow the aqueous mixture in the system downstream of the collector; an analyzer coupled to the pump and configured to allow the pump to flow the aqueous mixture through the analyzer and sense the aquatic organisms flowing in the aqueous mixture through the analyzer and allow discharge of the aqueous mixture from the analyzer; a processor coupled to the analyzer and configured to recognize and classify the aquatic organisms from input received from the analyzer; at least one of a data storage device to store data received from the processor; and a transmission system to send data received from the processor to a remote location.

The disclosure also provides an autonomous biomonitoring system for organisms in lotic ecosystems, comprising: a collector configured to collect an aqueous mixture having aquatic organisms from 1 to 50 mm in size as a largest cross sectional dimension; a pump coupled to the collector and configured to flow the aqueous mixture in the system downstream of the collector; an analyzer coupled to the pump and configured to sense the aquatic organisms flowing in the aqueous mixture; a processor coupled to the analyzer and configured to recognize and classify the aquatic organisms from input received from the analyzer; at least one of a data storage device to store data received from the processor; at least one of a data storage device to store data received from the processor; and a transmission system to send data received from the processor to a remote location.

### BRIEF DESCRIPTION OF THE SEVERAL VIEWS OF THE DRAWINGS

FIG. 1 is an illustrative example of an embodiment of an autonomous biomonitoring system of the invention.

FIG. 2 is a schematic block diagram of the embodiment of FIG. 1 with components and their relationship of the autonomous biomonitoring system of the invention.

FIG. 3 is an illustrative example a collector modified for continuous flow and coupling with an inline sediment sieve and a sediment trap.

FIG. 4 is a detail view of the example of the inline sediment sieve.

FIG. 5 is a detailed end view of the example of the inline sediment sieve in FIG. 4.

FIG. 6 is an illustrative example of a sediment trap as a separator of unwanted material from the flow stream.

FIG. 7 is an enlarged view of the staging unit of FIGS. 1 and 2.

FIG. 8 is an enlarged view of the flow through analyzer for continuous biomonitoring that is coupled with a processor with an optional power supply.

FIG. 9 is a schematic block diagram as an example of a second embodiment for variations from FIG. 2 with components and their relationship of an autonomous biomonitoring system of the invention.

FIG. 10 is a schematic illustration of a robot as a collector for obtaining samples according to the invention.

FIG. 11 is a schematic illustration of the robot shown in FIG. 10.

FIG. 12 is an illustration of the robot of FIG. 10 in operation.

### DETAILED DESCRIPTION

The Figures described above and the written description of specific structures and functions below are not presented to limit the scope of what Applicant has invented or the scope of the appended claims. Rather, the Figures and written description are provided to teach any person skilled in the art to make and use the inventions for which patent protection is sought. Those skilled in the art will appreciate that not all features of a commercial embodiment of the inventions are described or shown for the sake of clarity and understanding. Persons of skill in this art will also appreciate that the development of an actual commercial embodiment incorporating aspects of the present disclosure will require numerous implementation-specific decisions to achieve the developer's ultimate goal for the commercial embodiment. Such implementation-specific decisions may include, and likely are not limited to, compliance with system-related, business-related, government-related, and other constraints, which may vary by specific implementation or location, or with time. While a developer's efforts might be complex and time-consuming in an absolute sense, such efforts would be, nevertheless, a routine undertaking for those of ordinary skill in this art having benefit of this disclosure. It must be understood that the inventions disclosed and taught herein are susceptible to numerous and various modifications and alternative forms. The use of a singular term, such as, but not limited to, “a,” is not intended as limiting of the number of items. Further, the various methods and embodiments of the system can be included in combination with each other to produce variations of the disclosed methods and embodiments. Discussion of singular elements can include plural elements and vice-versa. References to at least one item may include one or more items. Also, various aspects of the embodiments could be used in conjunction with each other to accomplish the understood goals of the disclosure. Unless the context requires otherwise, the term “comprise” or variations such as “comprises” or “comprising,” should be understood to imply the inclusion of at least the stated element or step or group of elements or steps or equivalents thereof, and not the exclusion of a greater numerical quantity or any other element or step or group of elements or steps or equivalents thereof. The term “coupled,” “coupling,” “coupler,” and like terms are used broadly herein and may include any method or device for securing, binding, bonding, fastening, attaching, joining, inserting therein, forming thereon or therein, communicating, or otherwise associating, for example, mechanically, magnetically, electrically, chemically, operably, directly or indirectly with intermediate elements, one or more pieces of members together and may further include without limitation integrally forming one functional member with another in a unity fashion. The coupling may occur in any direction, including rotationally. The device or system may be used in a number of directions and orientations. The order of steps can occur in a variety of sequences unless otherwise specifically limited. The various steps described herein can be combined with other steps, interlineated with the stated steps, and/or split into multiple steps. Some elements are nominated by a device name for simplicity and would be understood to include a system or a section, such as a controller would encompass a processor and a system of related components that are known to those with ordinary skill in the art and may not be specifically described. Various examples are provided in the description and figures that perform various functions and are non-limiting in shape, size, description, but serve as illustrative structures that can be varied as would be known to one with ordinary skill in the art given the teachings contained herein.

The present disclosure provides an autonomous biomonitoring system in lotic ecosystems, such as streams, for small aquatic organisms (1 mm-50 mm), including benthic macroinvertebrates. A central design concept of autonomy is capturing organisms from their natural habitat and entraining them through an analyzer with an imaging unit. In at least one embodiment, the system provides a collector for an aqueous mixture that includes water and potential aquatic organisms that can flow through a filter to a separator that is coupled to a pump that discharges aqueous mixture into a staging unit with a staging tank. The staging tank is coupled to a flow-through analyzer that includes a scanner, electronic processor, and a database of characteristics of target aquatic organisms to analyze the aqueous mixture as it flows through the analyzer to determine types and quantities of any present aquatic organisms. The mixture can be discharged into the lotic ecosystem. In at least one embodiment, the system allows continuous monitoring over a period of time. The system can transmit real time data to a remote location for data accumulation. The system can further provide remote movement of the collector for sampling at multiple remote locations. The autonomous biomonitoring system provides data collection that heretofore has been unrealized as a significant shift in paradigm for sampling of streams. The invention can provide sampling in the field of aquatic organisms, particularly benthic macroinvertebrate and larval fish sampling, sample processing, and sample identification towards autonomy to provide higher volumes of information that is more representative of the actual conditions in the lotic ecosystem.

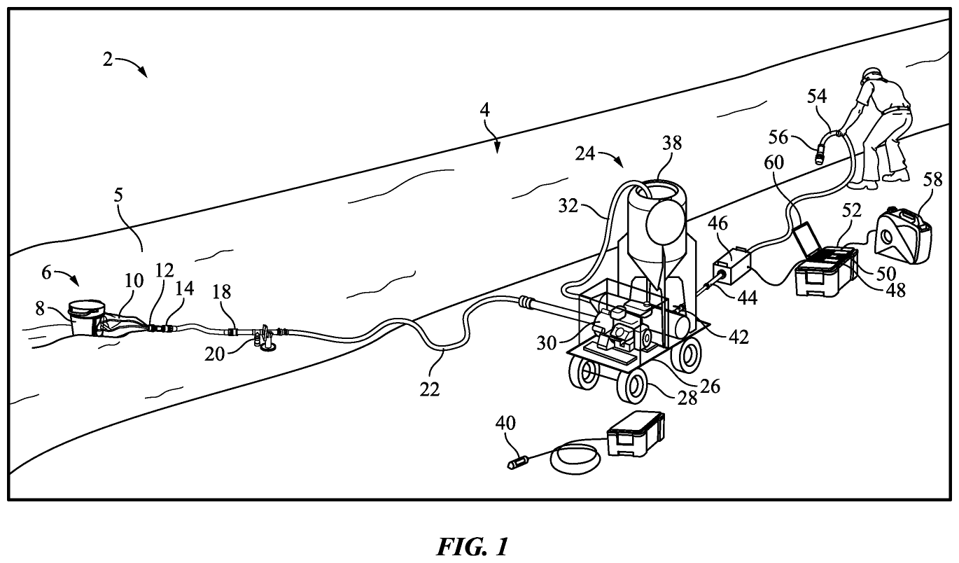

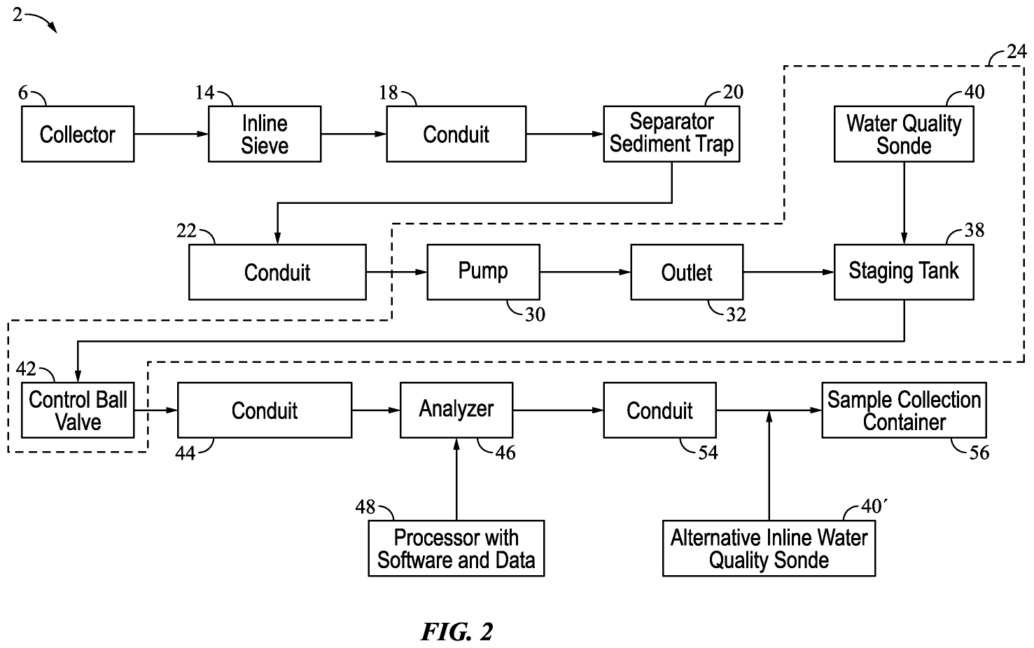

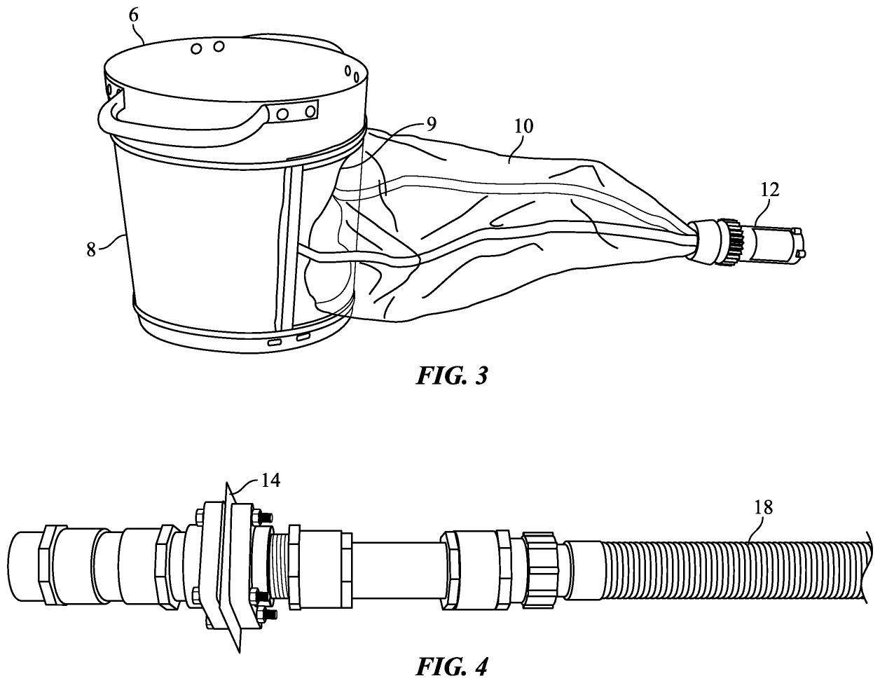

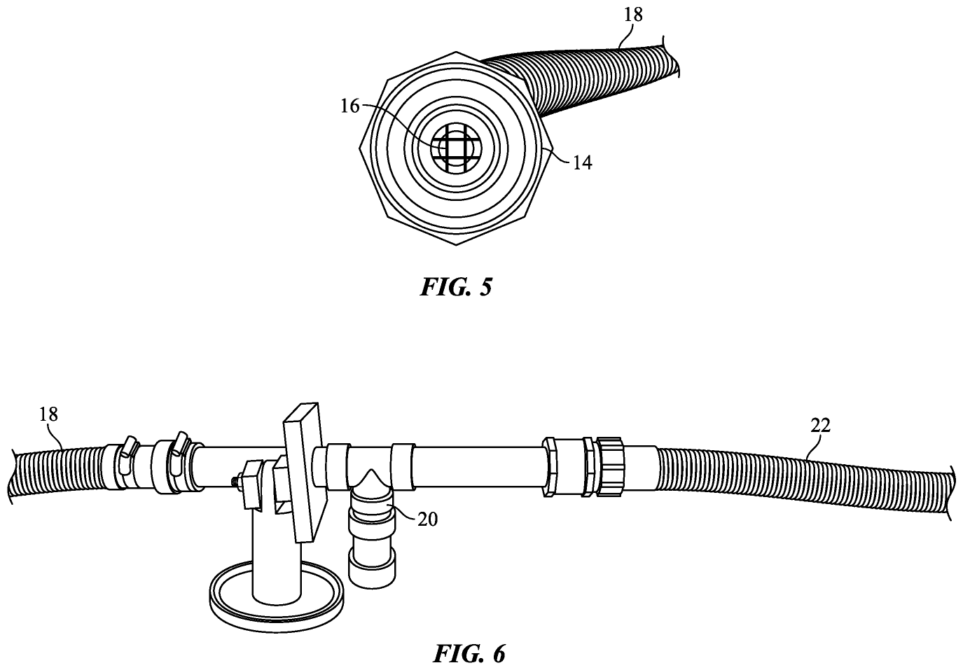

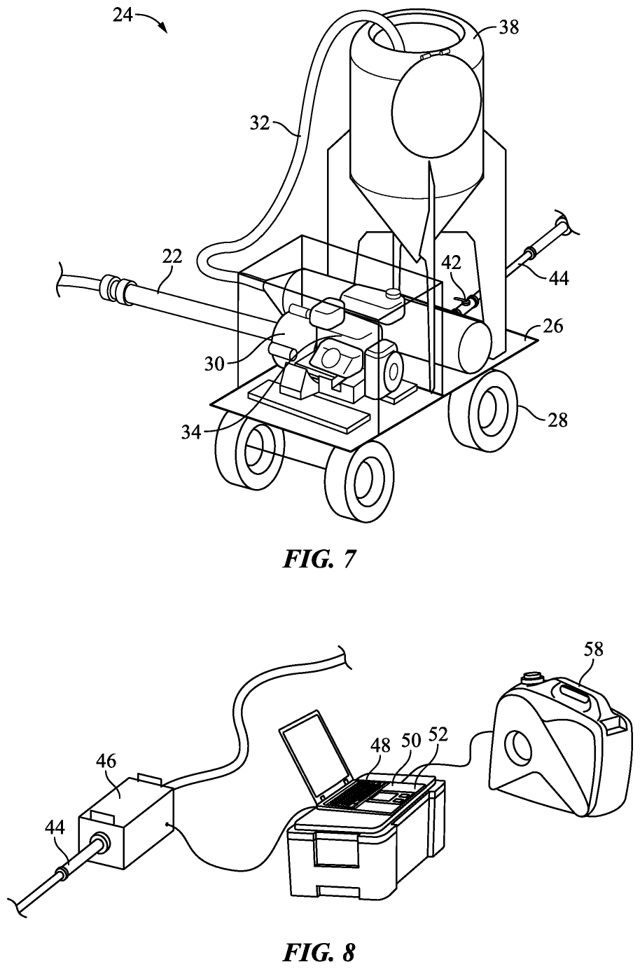

FIG. 1 is an illustrative example of an embodiment of an autonomous biomonitoring system of the invention. FIG. 2 is a schematic block diagram of the embodiment of FIG. 1 with components and their relationship of the autonomous biomonitoring system of the invention. FIG. 3 is an illustrative example a collector modified for continuous flow and coupling with an inline sediment sieve and a sediment trap. FIG. 4 is a detail view of the example of the inline sediment sieve. FIG. 5 is a detailed end view of the example of the inline sediment sieve in FIG. 4. FIG. 6 is an illustrative example of a sediment trap as a separator of unwanted material from the flow stream. FIG. 7 is an enlarged view of the staging unit of FIGS. 1 and 2. FIG. 8 is an enlarged view of the flow through analyzer for continuous biomonitoring that is coupled with a processor with an optional power supply.

The system 2 is shown in an illustrated embodiment with separate components. It is understood that in other embodiments many if not all of the components could be combined in a more compact form factor. However, the illustrated embodiment shows various components and their functions that can be included in other embodiments in various forms. Starting with the sampling initiation, a collector 6 has an inlet 8 to allow a flow of an aqueous mixture from a lotic ecosystem 4, such as a stream, into the collector. The aqueous mixture can include debris, sediment, gravel, aquatic organisms, and water. The collector can also be formed with an end open to allow inserting of the collector into a substrate 5, such as a stream bed, of the aqueous ecosystem 4 to disturb the substrate and uncover benith organisms for inclusion in the sampling. The collector 6 further includes an outlet 9 that can be coupled with a sieve 10 extending downstream of the collector in operation. The sieve 10 can have a mesh of sized openings to allow water to be released from the aqueous mixture back into the aqueous ecosystem while retaining objects larger than the sized openings to reduce volume of the aqueous mixture in the system 2 for analyzing. The sieve 10 can direct the reduced aqueous mixture into an adapter outlet 12 that is configured to be coupled with downstream system components. For example, a coarse inline filter 14 with a screen 16 can be coupled with the outlet 12 to remove relatively large objects that are not relevant to the organisms to be monitored. The inline filter 14 can be coupled to a conduit 18 to transporting the remaining aqueous mixture to downstream components. The conduit 18 can be rigid or flexible, such as a pipe or a hose, including a corrugated hose for flexibility and low footprint storage, or other flow channel. The conduit 18 can be coupled to a separator in the form of a sediment trap 20. The sediment trap 20 can assists in separating suspended sediment, gravel, and other particulates that may have a greater mass than the desired subject of analysis by allowing heavier objects to drop into the trap by gravity. The trap can be opened to periodically flush the objects. The sediment trap can be coupled to a conduit 22 to flow the aqueous mixture into a staging unit 24. The staging unit 24 can include a frame 26 with wheels 28 for portability, a pump 30 configured to pump the aqueous mixture, and a staging tank 38. The pump 30 can be a suction pump to pull the aqueous mixture through the conduit 22. The pump 30 includes a driver 34 to operate the pump, such as an electrical motor or combustion engine. Depending of the pump flow rate compared to the flow rate for sampling through the analyzer, a staging tank 38 coupled an outlet 32 of the pump may assist in buffering the flow prior to the analyzer, as shown in this embodiment. The staging tank 38 can be conical to help stage or otherwise buffer the flow into the analyzer. A value 42 and a conduit 44 can be coupled to the analyzer 46 from the pump or staging tank. Optionally, a water quality sonde 40 can be mounted in the container for additional data collection on the water quality that may affect the viability of the organisms. In another embodiment, the pump can discharge the aqueous mixture through the conduit 44 without the staging tank to the analyzer, in which case the sonde 40 may be inline with a conduit before or after the analyzer. The flow into the analyzer can be substantially continuous, either directly from the pump or from the staging tank.

The analyzer 46 is configured as an optical imaging and classification (OIC) system and is coupled with a processor 48. The processor can be a standalone computer coupled to the analyzer or a specially programmed processor integral with the analyzer. The processor can be coupled with a data storage device 50 having a database 52, in at least one embodiment. In at least one embodiment, the analyzer 46 provide a flow-through path with a sealed viewing port. Without limitation, characteristics for sorting can include physical attributes, such as shapes, sizes, colors, transparency, and weight. Non-physical attributes can include electrical resistivity and magnetic field changes with passage through analyzer. Other criteria are possible. A sensor (not shown) of the analyzer can include a high resolution optical imaging device that can view the aqueous mixture flowing by the viewing port to create a digital image of the flow. The digital imaging is sent to the processor for processing to recognize and classify the small aquatic organisms in the aqueous mixture flow based on database information. The processor can record the type and substantially the amount of the organisms relative to time and date. The processor can send the data to the data storage device for later downloading. In lieu of or in addition to the data storage device, the system can include a transmitter 60 (or transceiver) to send the data advantageously real time to a remote location. An artificial intelligence program can train the analyzer with the OIC system to adapt the recognition to a variety of the organisms for accurate classification. The aqueous mixture can flow out of the analyzer 46 through a conduit 54 to return, if desired, to the aqueous ecosystem. For quality testing and periodic validation of the analyzer results, one or more individual samples may be collected in a sample collector 56 for later analysis, such as at a laboratory.

A power supply 58 can provide remote power to the pump, analyzer, and processor. The power supply can be fuel, battery, solar, or even wind powered, depending on the location and needs. Advantageously, the power supply provides sufficient power for at least the desired monitoring period.

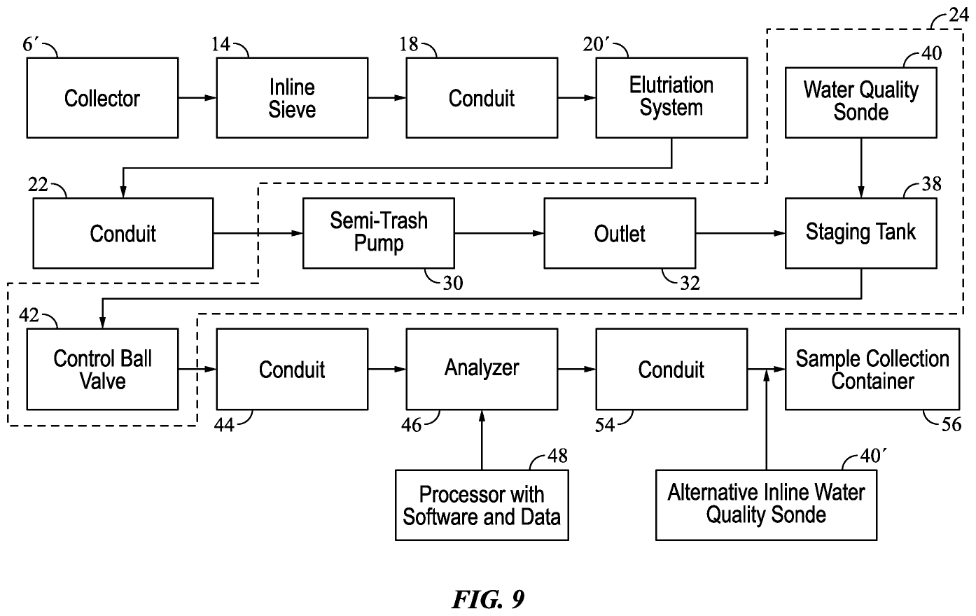

FIG. 9 is a schematic block diagram as an example of a second embodiment for variations from FIG. 2 with components and their relationship of an autonomous biomonitoring system of the invention. In this embodiment, the collector 6′ can be motorized for movement around the stream, such as in a submerged location as in the stream. The collector 6′ can include a mobile robot that can be preprogrammed in an array of movement, or with directed movement based on other criteria, such as slope, depth of penetration into the substrate of the aqueous ecosystem, and other criteria. The term “robot” is used broadly and includes mobility devices that can be controlled in movement, including remotely through wireless transmissions, manually by direct operator control, preset or programmed for an operation, self-directed based on designed or artificial intelligence sensory input, and other methods of directing movement. Further, the collector can include a GPS system to allow remote control based on remote monitoring of the collector location. The embodiment can also include a separator in the form of an elutriation system 20′ to separate objects and organisms in the aqueous mixture generally in a rotational flow based on for example size, shape, and density. The smaller objects and organisms generally flow to the top of the resulting flow. The embodiment can alternatively include the sonde 40′ inline with the aqueous mixture flow, such as downstream of the analyzer. Other components are similar as shown in FIG. 2 and described above.

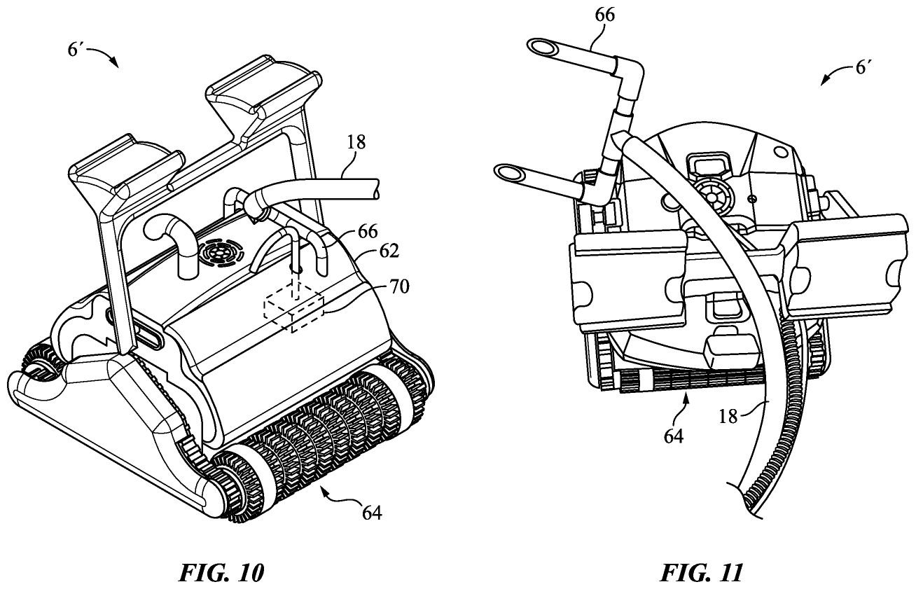

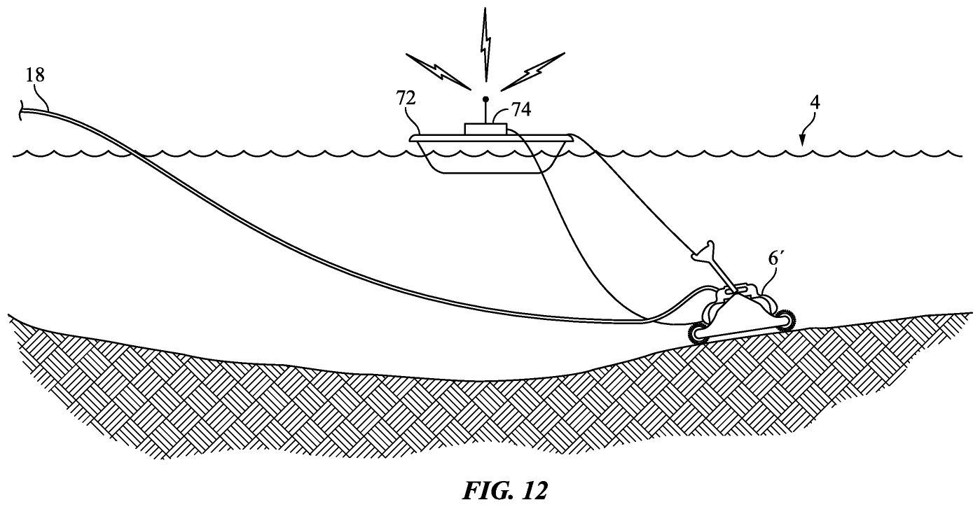

FIG. 10 is a schematic illustration of a robot as a collector for obtaining samples according to the invention. FIG. 11 is a schematic illustration of the robot shown in FIG. 10. FIG. 12 is an illustration of the robot of FIG. 10 in operation. The collector 6′ in the form of a robot can include a body 62 having one or more motors (not shown) for a mobility portion 64. For example, the mobility portion 64 can include a drive system and traction rollers to move the robot along the stream or other lotic surface. The body 62 can include a suction system 66 to receive a sampling portion of the lotic ecosystem having aquatic organisms by, for example, using suction from the pump 30, previously described and shown in FIGS. 1 and 7. Further, by motion of mobility portion 64, the robot can disturb the sediments on the surface of the stream bed, but also can disturb sediments below the first layer of substrate, for example if the sediments are smaller than medium size gravel.

The conduit 68 can be coupled to the body to receive the sampling portion for transfer ultimately to the pump. One or more sieves, filters, and/or separators (not shown) can be used as described above. Further, movement of the robot can be controlled through a control cable or wirelessly, such as through an antenna extending above the lotic ecosystem sufficiently to receive and transmit signals to the controller 70. A location of the robot can be tracked by a location device 74, such as a GPS transmitter. The location device may benefit from being above the lotic ecosystem and can be, for example, towed in a floatation device 72 with the robot.

Other and further embodiments utilizing one or more aspects of the inventions described above can be devised without departing from the disclosed invention as defined in the claims. For example, various types of robots can be used, including robots independently operable from a collector to move the collector, different types of collectors, pumps, staging tanks, if any, and other variations that may be appropriate to specific lotic ecosystem conditions. Other embodiments can include various parameters, user interface screens, various types of output and input, and other variations within the scope of the claims.

The invention has been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicant, but rather, in conformity with the patent laws, Applicant intend to protect fully all such modifications and improvements that come within the scope of the following claims.

What is claimed is:

1. An autonomous biomonitoring system for organisms in a lotic ecosystem, comprising:
a collector configured to collect an aqueous mixture having aquatic organisms;
a pump coupled to the collector and configured to flow the aqueous mixture in the system downstream of the collector;
an analyzer coupled to the pump and configured to allow the pump to flow the aqueous mixture through the analyzer and sense the aquatic organisms flowing in the aqueous mixture through the analyzer and allow discharge of the aqueous mixture from the analyzer;
a processor coupled to the analyzer and configured to recognize and classify the aquatic organisms from input received from the analyzer;
at least one of a data storage device to store data received from the processor; and
a transmission system to send data received from the processor to a remote location.

2. The system of claim 1, wherein the aquatic organisms comprise benthic macroinvertebrates.

3. The system of claim 1, wherein the analyzer comprises an optical sensor configured to view the aquatic organisms flowing in the aqueous mixture and provide the input to the processor.

4. The system of claim 1, wherein the pump is a suction pump disposed downstream of the collector and an output of the pump flows the aqueous mixture from the collector to the analyzer.

5. The system of claim 1, further comprising a staging tank disposed between the collector and the analyzer configured to receive of the pump output prior to the analyzer and buffer an aqueous mixture flow into the analyzer.

6. The system of claim 1, further comprising a power supply configured to supply power to at least one of the analyzer and the processor.

7. The system of claim 1, wherein the collector comprises a tube having at least one open end configured to be placed into a substrate of a lotic ecosystem, an side inlet, and a side outlet, the side outlet being coupled to a sieve configured to allow water to flow through the sieve and restrict passage of the aquatic organisms through the sieve.

8. The system of claim 1, wherein the collector comprises a robot configured to move based on instructions to different locations of a substrate of the lotic ecosystem, wherein the instructions comprise at least one of preprogrammed instructions and instructions transmitted from a remote location.

9. The system of claim 8, wherein the locations are based on Global Positioning System coordinates.

10. The system of claim 1, further comprising a water quality sonde configured to sense water quality of the aqueous mixture.

11. The system of claim 1, further comprising at least one of a filter and a separator configured to reduce material other than water and aquatic organisms from the aqueous mixture.

12. An autonomous biomonitoring system for organisms in lotic ecosystems, comprising:
a collector configured to collect an aqueous mixture having aquatic organisms from 1 to 50 mm in size as a largest cross sectional dimension;
a pump coupled to the collector and configured to flow the aqueous mixture in the system downstream of the collector;
an analyzer coupled to the pump and configured to sense the aquatic organisms flowing in the aqueous mixture;
a processor coupled to the analyzer and configured to recognize and classify the aquatic organisms from input received from the analyzer;
at least one of a data storage device to store data received from the processor;
at least one of a data storage device to store data received from the processor; and
a transmission system to send data received from the processor to a remote location.
